SEQUENCE LISTING (11 sequences)

SEQ ID NO: 1 - DNA, 130 bp, adeno-associated virus
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 2 - DNA, 14 bp, Artificial Sequence
  Synthesized
gcggccgcacgcgt

SEQ ID NO: 3 - DNA, 365 bp, Artificial Sequence
  Synthesized
tttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttgcttatgtaaaccagggcgcctataaaagagtgctgattttttgagtaaacttcaattccacaacacttttgtcttataccaactttccgtaccacttcctaccctcgtaaa

SEQ ID NO: 4 - DNA, 27 bp, Homo sapiens
cctgccgccaagagagtgaagctggac

SEQ ID NO: 5 - DNA, 3159 bp, Artificial Sequence
  Synthesized
ggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaactcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaaggga

SEQ ID NO: 6 - DNA, 66 bp, Artificial Sequence
  Synthesized
ggaagcggagctactaacttcagcctgctgaagcaggctggagacgtggaggagaaccctggacct

SEQ ID NO: 7 - DNA, 747 bp, Artificial Sequence
  Synthesized
atgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaa

SEQ ID NO: 8 - DNA, 49 bp, Artificial Sequence
  Synthesized
aataaaatatctttattttcattacatctgtgtgttggttttttgtgtg

SEQ ID NO: 9 - DNA, 141 bp, adeno-associated virus
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 10 - DNA, 7111 bp, Artificial Sequence
  Synthesized
cctgcaggcagctgcgcgctcgctcgctcactgaggccgcccgggcgtcgggcgacctttggtcgcccggcctcagtgagcgagcgagcgcgcagagagggagtggccaactccatcactaggggttccttttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttgcttatgtaaaccagggcgcctataaaagagtgctgattttttgagtaaacttcaattccacaacacttttgtcttataccaactttccgtaccacttcctaccctcgtaaacaccggtgccaccatgcctgccgccaagagagtgaagctggacggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaactcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggatccggacctgccgccaagagagtgaagctggacggaagcggagctactaacttcagcctgctgaagcaggctggagacgtggaggagaaccctggacctatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaataaaataaaatatctttattttcattacatctgtgtgttggttttttgtgtgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 11 - DNA, 7125 bp, Artificial Sequence
  Synthesized
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgttttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttgcttatgtaaaccagggcgcctataaaagagtgctgattttttgagtaaacttcaattccacaacacttttgtcttataccaactttccgtaccacttcctaccctcgtaaacaccggtgccaccatgcctgccgccaagagagtgaagctggacggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaactcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggatccggacctgccgccaagagagtgaagctggacggaagcggagctactaacttcagcctgctgaagcaggctggagacgtggaggagaaccctggacctatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaataaaataaaatatctttattttcattacatctgtgtgttggttttttgtgtgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

(19) United States
(12) Patent Application Publication
(10) Pub. No.: US 2023/0052011 A1
(43) Pub. Date: Feb. 16, 2023

(54) REGULATABLE EXPRESSION SYSTEMS

(71) Applicant: CRISPR THERAPEUTICS AG, Zug (CH)

(72) Inventors: Seshidhar Reddy Police, Cambridge, MA (US); Kyungah Maeng, Cambridge, MA (US)

(73) Assignee: CRISPR THERAPEUTICS AG, Zug (CH)

(21) Appl. No.: 17/782,505

(22) PCT Filed: Dec. 1, 2020
(86) PCT No.: PCT/IB2020/061297

§ 371 Date: Jun. 3, 2022

Related U.S. Application Data
(60) Provisional application No. 62/943,665, filed on Dec. 4, 2019.

(51) Int. Cl. C12N 15/63; C12N 15/69; C12N 15/86; C12N 9/22

(52) U.S. Cl. CPC C12N 15/635; C12N 15/69; C12N 15/86; C12N 9/22; C12N 2750/14143

(57) ABSTRACT

Provided herein are regulatable expression systems and methods of using said regulatable expression systems to express proteins of interest. The regulatable expression systems comprise a unidirectional regulatable promoter operably linked to a single transcription unit encoding a protein of interest, a ribosome skip, and a transactivator protein.

### SEQUENCE STATEMENT

This application contains a Sequence Listing that has been submitted in ASCII format via EFS-Web and is hereby incorporated by reference in its entirety. The ASCII copy, created on Nov. 30, 2020, is named 100867-675086-CT126-PCT-SequenceListing_ST25.txt, and is about 26,000 bytes in size.

### FIELD

The present disclosure is directed to regulatable expression systems and methods of using said regulatable expression systems to express proteins of interest.

### BACKGROUND

Gene therapy aims to treat or prevent diseases through the use of gene delivery systems. A key issue in successfully implementing gene therapies is the ability to regulate gene expression very tightly and consistently when needed. For example, it is desirable to turn gene expression “on” or “off” quickly and effectively, e.g., via contact with a regulator compound. Another challenge is that all regulatable expressions systems developed to date allow for leaky gene expression in the off state. This can be problem when the gene products are immunogenic or exert untoward effects if expressed long term. Thus, there is a need for regulatable gene delivery systems that are very responsive to the regulator compound and have reduced leaky expression.

### SUMMARY

Among the various aspects of the present disclosure in the provision of regulatable expression systems, wherein the regulatable expression systems are all-in-one systems.

In some aspects, the present disclosure provides a nucleic acid comprising a unidirectional regulatable promoter operably linked to a transcription unit that encodes a protein of interest, a ribosome skip, and a transactivator protein. In some instances, the transcription unit comprises from 5′ to 3′ sequence encoding the protein of interest, sequence encoding the ribosome skip, and sequence encoding the transactivator protein.

In some embodiments, the unidirectional regulatable promoter is a tetracycline-dependent promoter. In some instances, the unidirectional regulatable promoter comprises a plurality of tetracycline operator (tetO) sequences located upstream of a minimal constitutive eukaryotic promoter. For example, the unidirectional regulatable promoter can comprise from two to ten tetO sequences. In various embodiments, the minimal constitutive eukaryotic promoter can be a minimal cytomegalovirus (CMV) promoter, a minimal elongation factor 1 (EF1) alpha promoter, or a minimal Simian virus 40 (SV40) promoter. In some embodiments, the unidirectional regulatable promoter comprises seven tetO sequences located upstream of a minimal CMV promoter.

In some embodiments, the protein of interest encoded by the nucleic acid can be a recombinant protein or a therapeutic protein. In specific embodiments, the protein of interest can be a CRISPR protein, such as, for example, a Cas9 protein, a Cpf1 protein, a Cas13 protein, a Cas14 protein, a CasX protein, or a CasY protein. In some aspects, the CRISPR protein can have less than about 1200 amino acids. In some embodiments, the CRISPR protein can be Staphylococcus aureus Cas9, Neisseria meningitidis Cas9, Campylobacter jejuni Cas9, or a variant having at least 90% sequence identity to said Cas9 protein. In some embodiments, the CRISPR protein can be a CRISPR nuclease, a CRISPR nickase, or a nuclease deficient CRISPR variant. In some embodiments, the sequence encoding the CRISPR protein can be codon optimized for expression in a mammalian cell. In some embodiments, the CRISPR protein can be linked to at least one nuclear localization signal (NLS), wherein the at least one NLS can be located at or within 50 amino acids of the amino terminus and/or at or within 50 amino acids of the carboxy terminus of the CRISPR protein.

In some embodiments, the ribosome skip encoded by the nucleic acid can be a 2A sequence family member. In some embodiments, the transactivator protein encoded by the nucleic acid can be a variant of a reverse tetracycline transactivator (rtTA) protein that is linked to at least one activation domain. In some aspects, the at least one activation domain can be a VP16 activation domain or variant thereof. In some embodiments, the at least one activation domain can comprises more than one repeat of a minimal VP16 activation domain. In certain embodiments, the transactivator protein can be a variant of the rtTA protein that is linked to three repeats of a modified, minimal VP16 activation domain. In some embodiments, the sequence encoding the variant rtTA protein can be codon optimized for expression in a mammalian cell.

In some embodiments, the nucleic acid described above can further comprise a polyadenylation signal sequence at its 3′ end and/or an adeno-associated virus (AAV) inverted terminal repeat (ITR) at each end. In various instances, the nucleic acid can further comprise a spacer between the AAV ITR at its 5′ end and the unidirectional regulatable promoter. In some embodiments, the spacer can comprise from about 2 nucleotides or base pairs to about 30 nucleotides or base pairs. In some embodiments, the transcription unit of the nucleic acid cab further comprise sequence encoding another ribosome skip and a fluorescent protein.

Another aspect of the present disclosure encompasses an expression cassette comprising any one of the nucleic acids described above.

A further aspect of the present disclosure provides a vector comprising the expression cassette described above. In some embodiments, the plasmid vector has a sequence as set forth in SEQ ID NO: 10. In other embodiments, the plasmid vector has a sequence as set forth in SEQ ID NO: 11.

Still another aspect of the present disclosure provides, an AAV particle comprising any one of the nucleic acids described above and at least one capsid protein.

Yet another aspect of the present disclosure encompasses a mammalian cell comprising any one of the nucleic acids described herein, any one of the expression cassettes described herein, any one of the vectors described herein, or any one of the AAV particles described herein.

A further aspect of the present disclosure provides methods for expressing proteins of interest in cells, wherein a method comprises (a) introducing into a cell a regulatable expression system comprising a unidirectional regulatable promoter, wherein the regulatable expression system is provided by any one of the nucleic acids described herein, any one of the expression cassettes described herein, any one of the vectors described herein, or any one of the AAV particles described herein; and (b) exposing the cell to a promoter regulating agent. In some embodiments, the promoter regulating agent can be doxycycline. In some instances, basal expression of the protein of interest can be less than that from a regulatable expression system comprising a bidirectional regulatable promoter. In various embodiments, after exposure to the promoter regulating agent, the expression of the protein of interest from the regulatable system comprising the unidirectional regulatable promoter can be increased as compared to that from a regulatable expression system comprising a bidirectional regulatable promoter. In still other embodiments, after exposure to the promoter regulating agent, expression of the protein of interest from the regulatable system comprising the unidirectional regulatable promoter can be increased by at least 10-fold over basal expression.

In some embodiments, the protein of interest expressed by the cells can be a CRISPR protein. In various embodiments, the CRISPR protein can be a Cas9 protein, a Cpf1 protein, a Cas13 protein, a Cas14 protein, a CasX protein, or a CasY protein. In some aspects, the CRISPR protein can have less than about 1200 amino acids. In some embodiments, the CRISPR protein can be Staphylococcus aureus Cas9, Neisseria meningitidis Cas9, Campylobacter jejuni Cas9, or a variant having at least 90% sequence identity to said Cas9 protein. In some embodiments, the CRISPR protein can be a CRISPR nuclease, a CRISPR nickase, or a nuclease deficient CRISPR variant. In some embodiments, the sequence encoding the CRISPR protein can be codon optimized for expression in a mammalian cell. In some embodiments, the CRISPR protein can be linked to at least one nuclear localization signal (NLS), wherein the at least one NLS can be located at or within 50 amino acids of the amino terminus and/or at or within 50 amino acids of the carboxy terminus of the CRISPR protein.

### BRIEF DESCRIPTION OF THE DRAWINGS

FIG. 1 presents schematics of two single (unidirectional) promoter regulatable expression systems (pCTX-277 and pCTX-279) and a bidirectional promoter regulatable expression system (pCTX-276).

FIG. 2A shows the relative levels of SaCas9 protein expression from each expression system diagrammed in FIG. 1 from day 1 to day 6.

FIG. 2B presents Western blots showing the expression of SaCas9 and β-tubulin under various conditions.

FIG. 3A shows the relative expression of SaCas9 under various conditions in cells transfected with 100 ng plasmid.

FIG. 3B shows the relative expression of SaCas9 under various conditions in cells transfected with 250 ng plasmid.

### DETAILED DESCRIPTION

The present disclosure provides regulatable expression systems with increased sensitivity to a regulator compound and which have little or no leaky expression. The regulatable expressions systems disclosed herein comprise a unidirectional regulatable promoter operably linked to a single transcription unit encoding a protein of interest, a ribosome skip, and a transactivator protein. Upon expression, a single transcript is produced but due to a ribosome skip during translation, two separate proteins are produced. Additionally, the regulatable expressions systems disclosed herein are all-in-one systems, thereby facilitating their delivery to cells of interest. Also provided herein are methods of using the regulatable expression systems to express proteins of interest in cells of interest.

### (I) Regulatable Expression Systems

One aspect of the present disclosure encompasses expression systems comprising a unidirectional regulatable promoter operably linked to a single transcription unit encoding a protein of interest, a ribosome skip, and a transactivator protein, wherein the protein of interest and the transactivator protein are produced as separate proteins during translation due to the ribosome skip. These unidirectional regulatable promoter expression systems or cassettes have reduced levels of basal (leaky) expression and more tightly controlled regulated expression than bidirectional regulatable promoter expression systems.

(a) Unidirectional Regulatable Promoter

The regulatable expression systems disclosed herein comprise a (single) unidirectional regulatable promoter that drives expression of a downstream transcription unit. In general, the unidirectional regulatable promoter is a tetracycline (Tet)-dependent promoter, e.g., is regulated by doxycycline (Dox). More specifically, the unidirectional regulatable promoter is a Tet-On promoter. A regulatable Tet-dependent promoter comprises a plurality of tetracycline operator (tetO) sequences located upstream of a minimal constitutive eukaryotic promoter. A tetO sequence is a 19 bp binding element derived from an E. coli Tet operon. In some embodiments, the regulatable Tet-dependent promoter comprises two, three, four, five, six, seven, eight, nine, ten, or more than ten tetO sequences. A minimal constitutive eukaryotic promoter comprises the minimal elements necessary to drive gene expression. Suitable minimal constitutive eukaryotic promoters include minimal cytomegalovirus (CVM) promoter, minimal elongation factor 1 (EF1) alpha promoter, minimal Simian virus 40 (SV40) promoter, or an isolated TATA box. In some embodiments, the regulatable, unidirectional, Tet-dependent promoter comprises seven tetO sequences located upstream of a minimal CMV promoter. In specific embodiments, the regulatable, unidirectional, Tet-dependent promoter is a TRES promoter having the nucleotide sequence of SEQ ID NO: 3.

(b) Transcription Unit

The unidirectional regulatable promoter is operably linked to a transcription unit encoding the protein of interest, the ribosome skip, and the transactivator protein. In general, the transcription unit comprises from 5′ to 3′ sequence encoding the protein of interest, the ribosome skip, and the transactivator protein. There is no or very low levels of transcription from the transcription unit described herein in the absence of a promoter inducing agent.

Protein of Interest. The regulatable expression systems disclosed herein can be used for the expression of any protein of interest. For example, the protein of interest can be a recombinant protein, an engineered protein, a therapeutic protein, a fusion protein, and the like. In some instances, however, the size of the protein to be expressed can be a limitation. For example, in embodiments in which the regulatable expression system is an adeno-associated virus (AAV) system, the nucleotide sequence encoding the protein of interest can be no more than about 3.5 kb in length.

In some embodiments, the protein of interest can be a CRISPR protein derived from a prokaryotic clustered regularly interspersed short palindromic repeats (CRISPR) system. Suitable CRISPR proteins include CRISPR-associated (Cas) proteins such as Cas9 proteins, Cpf1 (or Cas12) proteins, Cas13 proteins (Zhang et al., Cell, 2018. 172(1):212-223.e17), Cas14 proteins (Harrington et al., Science, 2018, 362(6416):839-842), or CasX or CasY proteins (Burnstein et al., Nature, 2017, 542(7640):237-241). The CRISPR protein can be naturally occurring, a variant thereof, or a modified or engineered version thereof.

In some embodiments, the CRISPR protein can be Streptococcus pyogenes Cas9 (SpyCas9), Streptococcus. thermophilus CRISPR1 Cas9, Streptococcus thermophilus CRISPR 3 Cas9, Treponema denticola Cas9, Lachnospiraceae bacterium ND2006 Cpfl, Acidaminococcus sp. BV3L6 Cpfl, or variants having at least at least 85%, at least 90%, at least 92%, at least 94%, at least 96%, at least 98%, or at least 99% sequence identity to the protein.

In particular embodiments, the CRISPR protein contains less than about 1200 amino acids (aa). In specific embodiments, the Cas9 nuclease can be Staphylococcus aureus Cas9 (SauCas9; 1053 aa), Neisseria meningitidis Cas9 (NmeCas9; 1082 aa), Campylobacter jejuni Cas9 (CjeCas9; 984 aa), or a variant having at least at least 85%, at least 90%, at least 92%, at least 94%, at least 96%, at least 98%, or at least 99% sequence identity to said Cas9 protein. In other embodiments, the Cas9 nuclease can be Azospirillum B510 Cas9 (1168 aa), Campylobacter lari CF89-12 Cas9 (1103 aa), Corynebacter diphtheriae Cas9 (1084 aa), Eubacterium ventriosum Cas9 (1107 aa), Gluconacetobacter diazotrophicus Cas9 (1150 aa), Lactobacillus farciminis Cas9 (1126 aa), Neisseria cinerea Cas9 (1082 aa), Nitratitractor salsuginis DSM 16511 Cas9 (1132 aa), Parvibaculum lavamentivorans Cas9 (1037 aa), Roseburia intestinalis Cas9 (1128 aa), Sphaerochaeta globus Cas9 (1179 aa), Streptococcus pasteurianus Cas9 (1130 aa), Streptococcus thermophilus CRISPR1 (1121 aa), Streptococcus thermophilus LMD-9 Cas9 (1132 aa), or a variant having at least at least 85%, at least 90%, at least 92%, at least 94%, at least 96%, at least 98%, or at least 99% sequence identity to said Cas9 protein.

In some embodiments, the CRISPR protein can be a nuclease (e.g., cleaves both strands of a double strand sequence). In other embodiments, the CRISPR protein can be a nickase (e.g., cleave one strand of a double strand sequence). The nickase can be engineered via inactivation of one of the nuclease domains of a CRISPR nuclease. For example, the RuvC domain of a Cas9 protein can be inactivated by mutations such as D10A, D8A, E762A, and/or D986A, or the HNH of a Cas9 protein domain can be inactivated by mutations such as H840A, H559A, N854A, N856A, and/or N863A (with reference to the numbering system of Streptococcus pyogenes Cas9, SpyCas9) to generate a Cas9 nickase (e.g., nCas9). Comparable mutations in other CRISPR nucleases can generate nickases (e.g., nCpf1). In still other embodiments, the CRISPR protein can be a nuclease deficient variant (e.g., can comprise mutations in both the RuvC domain and the HNH domain). A nuclease deficient variant can be linked to an effector domain (e.g., transcriptional activation domain, base editing domain, and the like).

The CRISPR protein can be engineered by one or more amino acid substitutions, deletions, and/or insertions to have improved targeting specificity, improved fidelity, altered PAM specificity, decreased off-target effects, and/or increased stability. Non-limiting examples of one or more mutations that improve targeting specificity, improve fidelity, and/or decrease off-target effects include N497A, R661A, Q695A, K810A, K848A, K855A, Q926A, K1003A, R1060A, and/or D1135E (with reference to the numbering system of SpyCas9).

The CRISPR protein generally is linked to at least one nuclear localization signal (NLS) at the or within about 50 amino acids of N-terminal end, at or within about 50 amino acids of the C-terminal end, or both. In specific embodiments, the CRISPR protein is linked to a NLS at each end. NLSs are well known in the art. For example, the NLS can be a c-Myc NLS, SV40 Large T-antigen NLS, nucleoplasmin NLS, or derivatives thereof. The linkage between the CRISPR protein and the NLS can be a direct or it can be indirect via an intervening linker sequence. Suitable linker sequences are well known in the art.

Typically, the nucleotide sequence encoding the CRISPR protein is codon optimized for expression in eukaryotic cells of interest. For example, the sequence can be codon optimized for expression in human cells.

In specific embodiments, the protein of interest can be a Cas9 nuclease having less than about 1200 amino acids that is flanked by an NLS at each end.

Ribosome Skip. The transcription unit that is linked to the unidirectional regulatable promoter also includes the ribosome skip sequence. In some embodiments, the ribosome skip sequence can encode a short peptide (˜20 aa) that prevents the ribosome from creating the peptide bond between a glycine and a proline at the C terminal end of the ribosome skip peptide. The ribosome pauses after the glycine, resulting in release of the nascent polypeptide chains. Translation resumes, with the proline becoming the first amino acid of a second polypeptide chain. This mechanism results in apparent co-translational cleavage of the polyprotein. A highly conserved sequence at the C-terminus of the ribosome skip peptide contributes to steric hindrance and ribosome skipping. In general, the ribosome skip peptide is a 2A sequence family member. Suitable 2A sequence family members include F2A, T2A, E2A, and P2A, wherein F2A is derived from foot-and-mouth disease virus 2A, T2A is derived from thosea asigna virus 2A, E2A is derived from equine rhinitis A virus, and P2A derived from porcine teschovirus-1 2A. In some embodiment, the ribosome skip peptide is P2A.

In other embodiments, the ribosome skip can be an internal ribosome entry sequence (IRES), which is an RNA element that allows for translation initiation in a cap-independent manner. The IRES, therefore, allows for the production of two separate proteins from the single transcription unit. IRES elements are well known in the art, e.g., can be derived from viral genome (e.g., picornavirus, aphthovirus, pestivirus IERS) or from cellular mRNAs (e.g., various growth factors, transcription factors, oncogenes, and the like).

Transactivator Protein. The transcription unit also comprises sequence encoding the transactivator protein, which binds to the plurality of tetO sequences in the Tet-On promoter in the presence of regulator (e.g., Dox). The transactivator protein, therefore, comprises a reverse tetracycline transactivator (rtTA) protein or a variant thereof. A rtTA protein comprises four amino acid changes relative to a TA protein, and a variant rtTA can further comprise one or more amino acid changes chosen from V9I, S12G, F67S, F86Y, or T171K. The nucleotide sequence coding the rtTA protein can be codon optimized for expression in mammalian (e.g., human) cells. In general, the rtTA protein of the transactivator protein is linked to at least one activation domain.

The at least one activation domain can be derived from VP16, p65, Gal4, Gcn1, SP1, c-jun, AP2, Oct-2, NTF-1, or other suitable transcription activator. In some embodiments, the activation domain comprises a plurality of repeats of a minimal VP16 activation domain, which comprises 12 amino acids. The minimal VP16 domain can be modified and comprise at least one amino change (e.g., A209T). In some embodiments, the transactivator protein can comprise a rtTA protein variant linked to three repeats of a modified, minimal VP16 activation domain. In specific embodiments, the DNA sequence encoding the transactivator protein can have the nucleotide sequence of SEQ ID NO: 7.

Optional Additional Sequences. In some embodiments, the transcription unit optionally can further comprise (downstream of the transactivator protein sequence) a second ribosome skip and sequence encoding a fluorescent protein, such that a third polypeptide chain can be produced during translation. The second ribosome skip peptide can be the same as or can be different from the first ribosome skip described above. Suitable fluorescent proteins include, without limit, green fluorescent proteins (e.g., GFP, eGFP, GFP-2, tagGFP, turboGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., BFP, EBFP, EBFP2, Azurite, mKalama1, GFPuv, Sapphire, T-sapphire), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), orange fluorescent proteins (e.g., mOrange, mKO, Kusabira-Orange, Monomeric Kusabira-Orange, mTangerine, tdTomato), or combinations thereof.

Polyadenylation Sequence. The transcription unit further comprises a polyadenylation signal sequence at its 3′ end such that the mature messenger RNA comprises a polyA tail. Suitable polyadenylation signals include synthetic human growth hormone (hGH), bovine growth hormone (bGH), SV40, and rabbit beta-globin (rbGlob).

(c) Flanking Sequences

In some embodiments, the nucleic acid comprising the unidirectional regulatable promoter linked to the transcription unit described above can be flanked by repeat sequences. In some embodiments, the repeat sequences can be lentiviral long terminal repeat (LTR) sequences, retroviral LTR sequences, or adenoviral inverted terminal repeat (ITR) sequences. In specific embodiments, the repeat sequences can be adeno-associated virus (AAV) inverted terminal repeats (ITRs).

The 5′ and 3′ ITRs flanking the nucleic acid described above can be derived from any natural or recombinant AAV serotype. The 5′ and 3′ ITRs can be derived from the same or different AAV serotypes. Non-limiting examples of suitable serotypes include AAV1, AAV10, AAV106.1/hu.37, AAV11, AAV114.3/hu.40, AAV 12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAVI-7/rh.48, AAVI-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb.1, AAV29.5/bb.2, AAV2G9, AAV-2-pre-miRNA-101, AAV3, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r11.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAV A3.3, AAV A3.4, AAVA3.5, AAV A3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu.1, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu.7, AAVhu.8, AAVhu.9, AAVhu.t19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC 8, AAVpi.1, AAVpi.2, AAVpi.3, AAVrh.10, AAVrh.12, AAVrh.13, AAVrh.13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, BNP61 AAV, BNP62 AAV, BNP63 AAV, bovine AAV, caprine AAV, Japanese AAV 10, true type AAV (ttAAV), UPENN AAV 10, AAV-LK16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, and/or AAV SM 10-8. In specific embodiments, the ITRs can be derived from AAV-8 or variant thereof.

(d) Optional Spacer Sequence

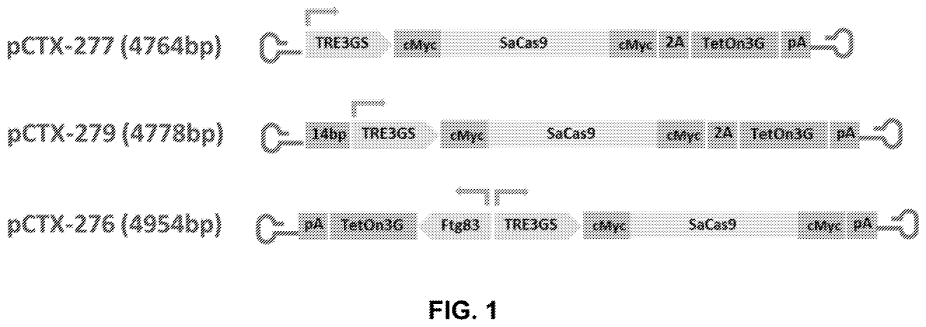

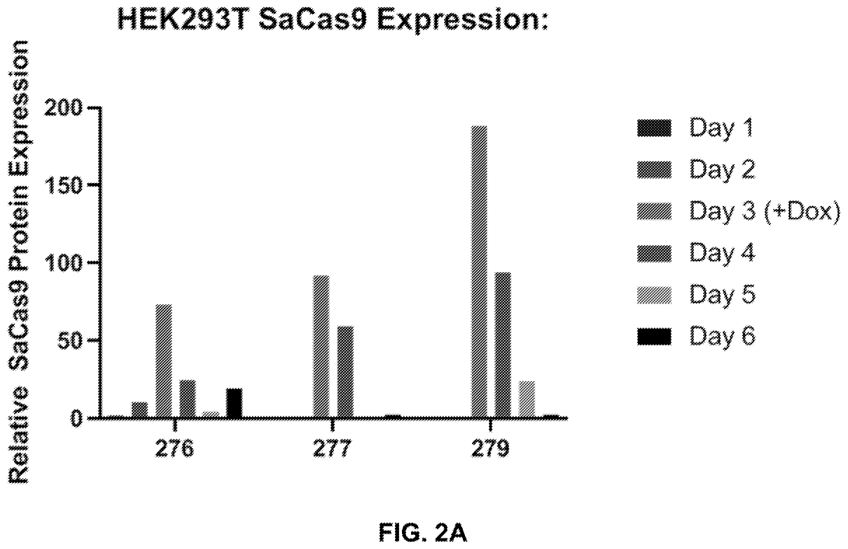

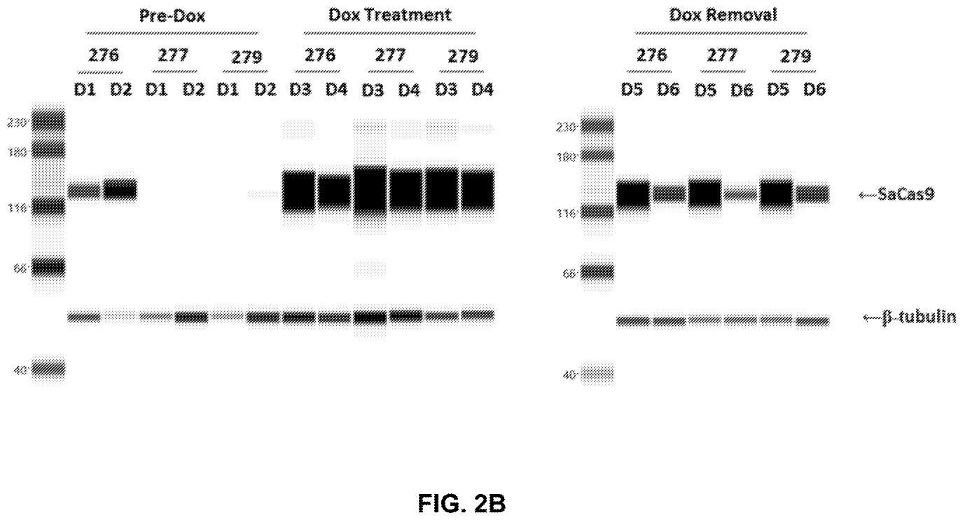

In certain embodiments, the regulatable expression system disclosed herein can further comprise a spacer sequence between the 5′ ITR and the unidirectional regulatable promoter (see FIG. 1). Surprisingly, the spacer allows for higher levels of regulated expression (see FIG. 2A). The spacer sequence can range in length from about 2 to about 30 nucleotides (or base pairs). In various embodiments, the length of the spacer can be 2, 3, 4, 5, 6, 7, 8, 9, 10, 11, 12, 13, 14, 15, 16, 17, 18, 19, 20, 21, 22, 223, 24, 25, 26, 27, 28, 29, or 30 nucleotides or base pairs. In specific embodiments, the spacer can be a 14 bp sequence.

(e) Specific Regulatable Expression Systems

In specific embodiments, the regulatable expression system comprises a Tet-On regulatable promoter comprising seven tetO sequence upstream of a minimal CMV promoter, wherein the Tet-On regulatable promoter is operably linked to a transcription unit encoding a CRISPR protein, e.g., a CRISPR nuclease having less than about 1200 amino acids that is flanked by a NLS at each end, a ribosome skip peptide of the 2A sequence family, a transactivator protein that comprises a variant rtTA protein linked to three repeats of the minimal VP16 activation domain, and a polyadenylation signal sequence, wherein the regulatable expression system is flanked by 5′ and 3′ AAV ITRs.

### (II) Recombinant AAV Particles

Another aspect of the present disclosure encompasses recombinant AAV (rAAV) particles (also called virions) comprising any of the expression systems described above in sections (I)(a) and (I)(b) that is flanked by AAV ITRs and which is encapsidated by at least one AAV capsid (Cap) protein. Typically, all the Cap proteins of an AAV are present in the rAAV particle. The Cap proteins can be wildtype AAV Cap proteins or can be variant AAV Cap proteins that may have altered and/or enhanced tropism towards one or more cell types. Means for producing rAAV particles are well known in the art.

### (Ill) Delivery Systems Comprising Regulatable Expression Cassettes

A further aspect of the present disclosure comprises delivery systems comprising the regulatable expression cassettes described above in section (I). Suitable delivery systems include viral vector delivery systems other than the AAV systems described above (e.g., lentiviral, adenoviral, retroviral, and the like), as well as non-viral delivery systems. In some embodiments, the non-viral delivery system can be plasmid-based systems. Suitable plasmid backbones are well known in the art. The plasmid vector can further comprise at least one origin of replication and/or at least one selectable marker sequence (e.g., antibiotic resistance genes) for propagation and selection in cells of interest. Additional information about vectors and use thereof can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001.

In one embodiment, the plasmid vector comprising a regulatable expression system has the sequence of SEQ ID NO: 10. In another embodiment, the plasmid vector comprising a regulatable expression system has the sequence of SEQ ID NO: 11.

### (IV) Cells Comprising Regulatable Expression Systems

Still another aspect of the present disclose comprises cells comprising the regulatable expression systems as described in section (I), rAAV particles as described in section (II), or vectors as described in section (III). In general, the cell is a eukaryotic cell, e.g., a mammalian cell. In specific embodiments, the cell can be a human cell.

In some embodiments, the cells can be in vitro (e.g., cell line cells, cultured cells, primary cells). In other embodiments, the cells can be ex vivo cells isolated from an organism. In still other embodiments, the cells can be in vivo cells within an organism.

In some embodiments, the cells may be stem cells (e.g., embryonic stem cells, fetal stem cells, amniotic stem cells, or umbilical cord stem cells). In certain embodiments, the stem cells may be adult stem cells isolated from bone marrow, adipose tissue, or blood. In still other embodiments, the cells may be induced pluripotent stem cells (e.g., human iPSCs).

In particular embodiments, the cells may be hematopoietic stem and progenitor cells (HSPCs) or hematopoietic stem cells (HSCs). HSPCs give rise to all blood cell types, including erythroid (erythrocytes or red blood cells (RBCs)), myeloid (monocytes and macrophages, neutrophils, basophils, eosinophils, megakaryocytes/platelets, and dendritic cells), and lymphoid (T-cells, B-cells, NK-cells). Blood cells are produced by the proliferation and differentiation of a very small population of pluripotent HSCs that also have the ability to replenish themselves by self-renewal. During differentiation, the progeny of HSCs progress through various intermediate maturational stages, generating multi-potential and lineage-committed progenitor cells prior to reaching maturity. Bone marrow (BM) is the major site of hematopoiesis in humans and, under normal conditions, only small numbers of HSPCs can be found in the peripheral blood (PB). Treatment with cytokines (in particular granulocyte colony-stimulating factor; G-CSF), some myelosuppressive drugs used in cancer treatment, and compounds that disrupt the interaction between hematopoietic and BM stromal cells can rapidly mobilize large numbers of stem and progenitors into the circulation. The cell surface glycoprotein CD34 is routinely used to identify and isolate HSPCs.

In other embodiments, the cells may be mesenchymal stem cells (e.g., multipotent stromal cells that can differentiate into a variety of cell types). Mesenchymal stem cells (MSCs) are adult stem cells found in the bone marrow, or isolated from other tissues such as cord blood, peripheral blood, fallopian tube, and fetal liver and lung. As multipotent stem cells, MSCs differentiate into multiple cell types including adipocytes, chondrocytes, osteocytes, and cardiomyocytes. Mesenchymal stem cells are a distinct entity to the mesenchyme, embryonic connective tissue, which is derived from the mesoderm and differentiates to form hematopoietic stem cells (HPCs).

In still other embodiments, the cells may be immune cells such as T cells, B cells, natural killer (NK) cells, NKT cells, mast cells, eosinophils, basophils, macrophages, neutrophils, or dendritic cells.

In further embodiments, the cells may be primary cells isolated directly from human or animal tissue. Non-limiting examples of suitable primary cells include adipocytes, astrocytes, blood cells (e.g., erythroid, lymphoid), chondrocytes, endothelial cells, epithelial cells, fibroblasts, hair cells, hepatocytes, keratinocytes, melanocyte, myocytes, neurons, osteoblasts, skeletal muscle cells, smooth muscle cells, stem cells, or synoviocytes.

In additional embodiments, the cells can be (immortalized) mammalian cell line cells. Non-limiting examples of suitable mammalian cell lines include human embryonic kidney cells (HEK293, HEK293T); human cervical carcinoma cells (HELA); human lung cells (W138); human liver cells (Hep G2); human U2-OS osteosarcoma cells, human A549 cells, human A-431 cells, and human K562 cells; Chinese hamster ovary (CHO) cells, baby hamster kidney (BHK) cells; mouse myeloma NS0 cells, mouse embryonic fibroblast 3T3 cells (NIH3T3), mouse B lymphoma A20 cells; mouse melanoma B16 cells; mouse myoblast C2C12 cells; mouse myeloma SP2/0 cells; mouse embryonic mesenchymal C3H-10T1/2 cells; mouse carcinoma CT26 cells, mouse prostate DuCuP cells; mouse breast EMT6 cells; mouse hepatoma Hepa1c1c7 cells; mouse myeloma J5582 cells; mouse epithelial MTD-1A cells; mouse myocardial MyEnd cells; mouse renal RenCa cells; mouse pancreatic RIN-5F cells; mouse melanoma X64 cells; mouse lymphoma YAC-1 cells; rat glioblastoma 9L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS7) cells; monkey kidney CVI-76 cells; African green monkey kidney (VERO-76) cells. An extensive list of mammalian cell lines may be found in the American Type Culture Collection catalog (ATCC, Manassas, Va.).

### (V) Methods for Expressing Proteins of Interest

Yet another aspect of the present disclosure encompasses methods for expressing proteins of interest, wherein the method comprises introducing into cells of interest a regulatable expression system or cassette as described in section (I), rAAV particles as described in section (II), or vector as described in section (III), and exposing the cells to a promoter regulating agent.

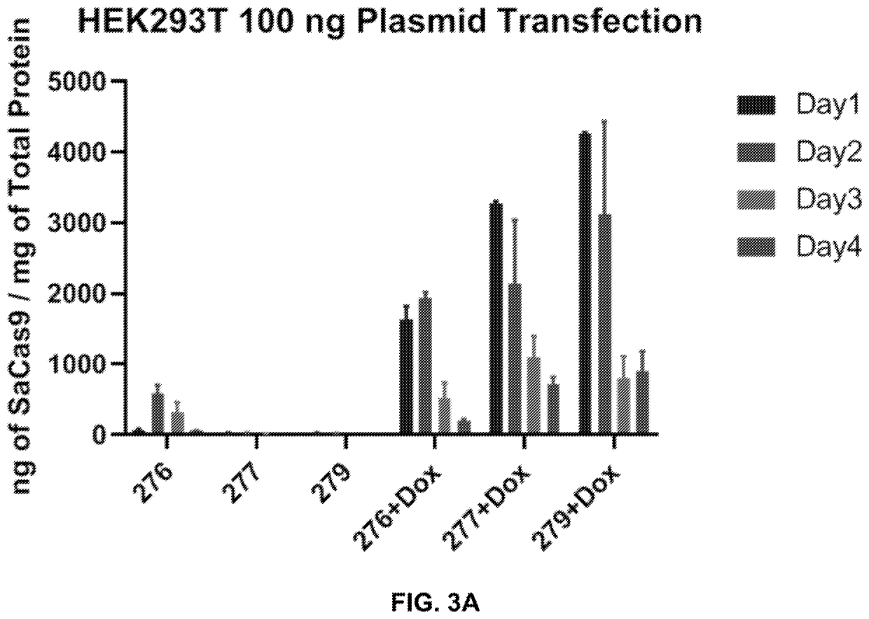

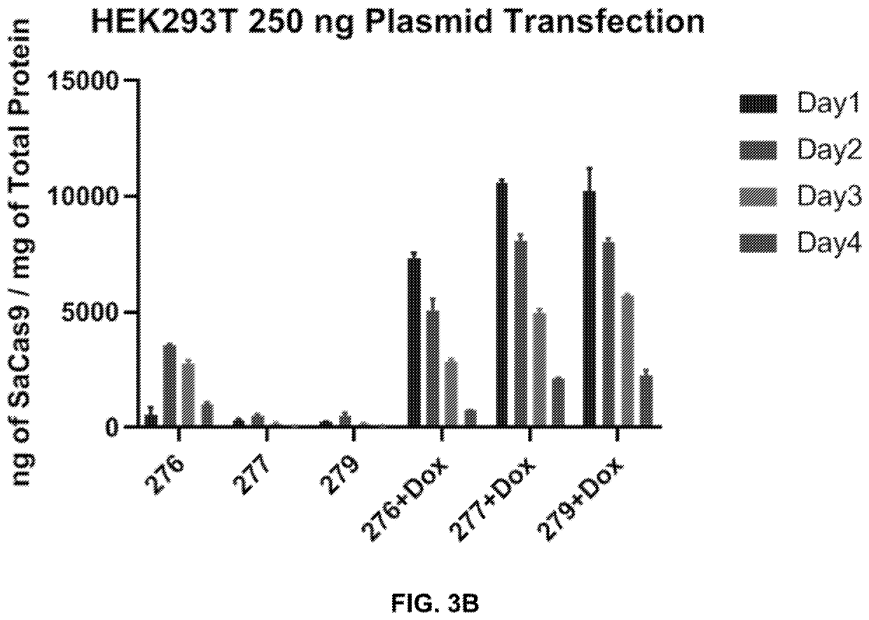

Basal or leaky expression of the protein of interest from the unidirectional regulatable promoter is reduced as compared to that from a regulatable expression system comprising a bidirectional regulatable promoter. In some embodiments, basal expression from the unidirectional regulatable promoter is below the limit of detection. In other embodiments, the level of basal expression from the unidirectional regulatable promoter is reduced at least 2-fold, at least 5-fold, at least 10-fold, at least 15-fold, or at least 20-fold relative to basal expression from a bidirectional regulatable promoter (see, e.g., FIG. 3B).

Regulated expression is induced upon exposure to a promoter regulating agent. In general, the promoter regulating agent is doxycycline (Dox). The concentration of Dox presented to the cells can and will vary depending, for example, on the desired level of expression of the protein of interest. That is, the level of expression is positively correlated with the level of Dox. In various embodiments, the concentration of Doc can range from about 0.1 ng/mL to about 1000 ng/mL. In certain embodiments, the concentration of Dox can range from about 10 ng/m L to about 100 ng/m L.

Upon exposure to Dox, the level of expression of the protein of interest from the unidirectional regulatable promoter can be increased by at least about 10-fold, at least about 30-fold, at least about 100-fold, at least about 300-fold, at least about 1000-fold, at least about 3000-fol, at least bout 10,000-fold, or at least about 30,000-fold over basal expression. Additionally, the level of expression of the protein of interest from the unidirectional regulatable promoter can be higher than expression from a regulatable expression system comprising a bidirectional regulatable promoter (see, e.g., FIGS. 2A-B, 3A-B).

Suitable cells are described above in section (IV). In embodiments in which the cells are in vitro, the cells are cultured under well-known conditions.

### (VI) Specific Compositions and Methods of the Disclosure

Accordingly, the present disclosure relates in particular to the following non-limiting compositions and methods.

In a first composition, Composition 1, the present disclosure provides a composition comprising a nucleic acid comprising a unidirectional regulatable promoter operably linked to a transcription unit, the transcription unit encoding a protein of interest, a ribosome skip, and a transactivator protein.

In another composition, Composition 2, the present disclosure provides a composition, as provided in Composition 1, wherein the transcription unit comprises from 5′ to 3′ sequence encoding the protein of interest, sequence encoding the ribosome skip, and sequence encoding the transactivator protein.

In another composition, Composition 3, the present disclosure provides a composition, as provided in Compositions 1 or 2, wherein the unidirectional regulatable promoter is a tetracycline-dependent promoter.

In another composition, Composition 4, the present disclosure provides a composition, as provided in any one of Compositions 1 to 3, wherein the unidirectional regulatable promoter comprises a plurality of tetracycline operator (tetO) sequences located upstream of a minimal constitutive eukaryotic promoter.

In another composition, Composition 5, the present disclosure provides a composition, as provided in Composition 4, wherein the unidirectional regulatable promoter comprises from two to ten tetO sequences.

In another composition, Composition 6, the present disclosure provides a composition, as provided in Compositions 4 or 5, wherein the minimal constitutive eukaryotic promoter is a minimal cytomegalovirus (CMV) promoter, a minimal elongation factor 1 (EF1) alpha promoter, or a minimal Simian virus 40 (SV40) promoter.

In another composition, Composition 7, the present disclosure provides a composition, as provided in any one of Compositions 4 to 6, wherein the unidirectional regulatable promoter comprises seven tetO sequences located upstream of a minimal CMV promoter.

In another composition, Composition 8, the present disclosure provides a composition, as provided in any one of Compositions 1 to 7, wherein the protein of interest encoded by the transcription unit is a recombinant protein or a therapeutic protein.

In another composition, Composition 9, the present disclosure provides a composition, as provided in any one of Compositions 1 to 8, wherein the protein of interest is CRISPR protein.

In another composition, Composition 10, the present disclosure provides a composition, as provided in Composition 9, wherein the CRISPR protein is a Cas9 protein, a Cpf1 protein, a Cas13 protein, a Cas14 protein, a CasX protein, or a CasY protein.

In another composition, Composition 11, the present disclosure provides a composition, as provided in Compositions 9 or 10, wherein the CRISPR protein has less than about 1200 amino acids.

In another composition, Composition 12, the present disclosure provides a composition, as provided in any one of Compositions 9 to 11, wherein the CRISPR protein is Staphylococcus aureus Cas9, Neisseria meningitidis Cas9, Campylobacter jejuni Cas9, or a variant having at least 90% sequence identity to said Cas9 protein.

In another composition, Composition 13, the present disclosure provides a composition, as provided in any one of Compositions 9 to 12, wherein the CRISPR protein is a CRISPR nuclease, a CRISPR nickase, or a nuclease deficient CRISPR variant.

In another composition, Composition 14, the present disclosure provides a composition, as provided in any one of Compositions 9 to 13, wherein sequence encoding the CRISPR protein is codon optimized for expression in a mammalian cell.

In another composition, Composition 15, the present disclosure provides a composition, as provided in any one of Compositions 9 to 14, wherein the CRISPR protein is linked to at least one nuclear localization signal (NLS).

In another composition, Composition 16, the present disclosure provides a composition, as provided in Composition 15, wherein the at least one NLS is located at or within 50 amino acids of the amino terminus and/or at or within 50 amino acids of the carboxy terminus of the CRISPR protein.

In another composition, Composition 17, the present disclosure provides a composition, as provided in any one of Compositions 1 to 16, wherein the ribosome skip encoded by the transcription unit is a 2A sequence family member.

In another composition, Composition 18, the present disclosure provides a composition, as provided in any one of Compositions 1 to 17, wherein the transactivator protein encoded by the transcription unit is a variant of a reverse tetracycline transactivator (rtTA) protein that is linked to at least one activation domain.

In another composition, Composition 19, the present disclosure provides a composition, as provided in Composition 18, wherein sequence encoding the variant rtTA protein is codon optimized for expression in a mammalian cell.

In another composition, Composition 20, the present disclosure provides a composition, as provided in Compositions 18 or 19, wherein the at least one activation domain is a VP16 activation domain or variant thereof.

In another composition, Composition 21, the present disclosure provides a composition, as provided in Composition 20, wherein the at least one activation domain comprises one or more repeats of a minimal VP16 activation domain.

In another composition, Composition 22, the present disclosure provides a composition, as provided in any one of Compositions 18 to 21, wherein the transactivator protein comprises the variant rtTA protein linked to three repeats of a modified, minimal VP16 activation domain.

In another composition, Composition 23, the present disclosure provides a composition, as provided in any one of Compositions 1 to 22, further comprising a polyadenylation signal sequence at its 3′ end.

In another composition, Composition 24, the present disclosure provides a composition, as provided in any one of Compositions 1 to 23, further comprising an adeno-associated virus (AAV) inverted terminal repeat (ITR) at each end/

In another composition, Composition 25, the present disclosure provides a composition, as provided in any one of Compositions 1 to 24, further comprising a spacer between the AAV ITR at its 5′ end and the unidirectional regulatable promoter.

In another composition, Composition 26, the present disclosure provides a composition, as provided in Composition 25, wherein the spacer comprises from about 2 nucleotides or base pairs to about 30 nucleotides or base pairs.

In another composition, Composition 27, the present disclosure provides a composition, as provided in any one of Compositions 1 to 26, wherein the transcription unit further encodes another ribosome skip and a fluorescent protein.

In another composition, Composition 28, the present disclosure provides an expression cassette comprising the nucleic acid as provided in any one of Compositions 1 to 27.

In another composition, Composition 29, the present disclosure provides a vector comprising the expression cassette as provided in Composition 28.

In another composition, Composition 30, the present disclosure provides a plasmid vector having a sequence as set forth in SEQ ID NO: 10.

In another composition, Composition 31, the present disclosure provides a plasmid vector having a sequence as set forth in SEQ ID NO: 11.

In another composition, Composition 32, the present disclosure provides an AAV particle comprising the nucleic acid as provided in any one of Compositions 1 to 27 and at least one capsid protein.

In another composition, Composition 32, the present disclosure provides a mammalian cell comprising the nucleic acid as provided in any one of Compositions 1 to 27, the expression cassette as provided in Composition 28, the vector as provided in any one of Compositions 29 to 31, or the AAV particle as provided in Composition 32.

In a first method, Method 1, the present disclosure provides a method for expressing a protein of interest in a cell, the method comprising (a) introducing into the cell a regulatable expression system comprising a unidirectional regulatable promoter, wherein the regulatable expression system is provided by the nucleic acid as provided in any one of Compositions 1 to 27, the expression cassette as provided in Composition 28, the vector as provided in any one of Compositions 29 to 31, or the AAV particle as provided in Composition 32; and (b) exposing the cell to a promoter regulating agent.

In another method, Method 2, the present disclosure provides a method, as provided in Method 1, wherein the promoter regulating agent is doxycycline.

In another method, Method 3, the present disclosure provides a method, as provided in Methods 1 or 2, wherein basal expression of the protein of interest from the regulatable expression system comprising a unidirectional regulatable promoter is less than that from a regulatable expression system comprising a bidirectional regulatable promoter.

In another method, Method 4, the present disclosure provides a method, as provided in any one of Methods 1 to 3, wherein, upon exposure to the promoter regulating agent, expression of the protein of interest from the regulatable expression system comprising a unidirectional regulatable promoter is increased as compared to that from a regulatable expression system comprising a bidirectional regulatable promoter.

In another method, Method 5, the present disclosure provides a method, as provided in any one of Methods 1 to 4, wherein, upon exposure to the promoter regulating agent, expression of the protein of interest from the regulatable expression system comprising a unidirectional regulatable promoter is increased by at least 10-fold over basal expression.

In another method, Method 6, the present disclosure provides a method, as provided in any one of Methods 1 to 5, wherein the protein of interest is a CRISPR protein.

In another method, Method 7, the present disclosure provides a method, as provided in Method 6, wherein the CRISPR protein is a Cas9 protein, a Cpf1 protein, a Cas13 protein, a Cas14 protein, a CasX protein, or a CasY protein.

In another method, Method 8, the present disclosure provides a method, as provided in Methods 6 or 7, wherein the CRISPR protein has less than about 1200 amino acids.

In another method, Method 9, the present disclosure provides a method, as provided in Methods 6 or 7, wherein the CRISPR protein is Staphylococcus aureus Cas9, Neisseria meningitidis Cas9, Campylobacter jejuni Cas9, or a variant having at least 90% sequence identity to said Cas9 protein.

### Definitions

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

As used herein, the terms “complementary” or “complementarity” refer to the association of double-stranded nucleic acids by base pairing through specific hydrogen bonds. The base paring may be standard Watson-Crick base pairing (e.g., 5′-A G T C-3′ pairs with the complementary sequence 3′-T C A G-5′). The base pairing also may be Hoogsteen or reversed Hoogsteen hydrogen bonding. Complementarity is typically measured with respect to a duplex region and thus, excludes overhangs, for example. Complementarity between two strands of the duplex region may be partial and expressed as a percentage (e.g., 70%), if only some (e.g., 70%) of the bases are complementary. The bases that are not complementary are “mismatched.” Complementarity may also be complete (i.e., 100%), if all the bases in the duplex region are complementary.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “heterologous” refers to an entity that is not endogenous or native to the cell of interest. For example, a heterologous protein refers to a protein that is derived from or was originally derived from an exogenous source, such as an exogenously introduced nucleic acid sequence. In some instances, the heterologous protein is not normally produced by the cell of interest. Protein.

The terms “nuclease” and “endonuclease” are used interchangeably herein, and refer to an enzyme that cleaves both strands of a double-stranded nucleic acid sequence.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The term “nucleotide” refers to deoxyribonucleotides or ribonucleotides. The nucleotides may be standard nucleotides (i.e., adenosine, guanosine, cytidine, thymidine, and uridine), nucleotide isomers, or nucleotide analogs. A nucleotide analog refers to a nucleotide having a modified purine or pyrimidine base or a modified ribose moiety. A nucleotide analog may be a naturally occurring nucleotide (e.g., inosine, pseudo uridine, etc.) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The term “sequence identity” as used herein, indicates a quantitative measure of the degree of identity between two sequences of substantially equal length. The percent identity of two sequences, whether nucleic acid or amino acid sequences, is the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website.

Where a range of values is provided, each value between the upper and lower ends of the range are specifically contemplated and described herein.

### EXAMPLES

The following examples illustrate various non-limiting embodiments of the present disclosure.

### Example 1. Unidirectional Promoter Tet-On Gene Expression Systems

Two unidirectional regulatable promoter (Tet-On) gene expression plasmids were constructed and tested. FIG. 1 summarizes construct design. The two expression cassettes (pCTX-277 and pCTX-279) comprise a single unidirectional promoter (TRE3GS) operably linked to a single transcription unit coding two proteins, (e.g., SaCas9 and Tet-On 3G transactivator protein) such that two separate proteins are synthesized during translation due to a ribosomal skip (e.g., 2A sequence). Table 1 identifies the elements and their locations in the two unidirectional regulatable promoter expression cassettes. The complete sequence of pCTX-277 is presented in SEQ ID NO: 10, and the complete sequence of pCTX-279 is presented in SEQ ID NO: 11.

TABLE 1
Elements of Single Promoter Expression Systems

| | pCTX-277 | pCTX-279 | |
|---|---|---|---|
| Element | Location (size) | Location (size) | SEQ ID NO: |
| Left AAV-ITR | 1-130 (130 bp) | 1-130 (130 bp) | 1 |
| Spacer | — | 131-144 (14 bp) | 2 |
| TRES promoter | 131-495 (365 bp) | 145-509 (365 bp) | 3 |
| cMyc NLS | 512-538 (27 bp) | 526-552 (27 bp) | 4 |
| SaCas9 | 545-3703 (3159 bp) | 559-3717 (3159 bp) | 5 |
| cMyc NLS | 3710-3736 (27 bp) | 3724-3750 (27 bp) | 4 |
| 2A sequence | 3737-3802 (66 bp) | 3751-3816 (66 bp) | 6 |
| TetOn3G | 3803-4549 (747 bp) | 3817-4563 (747 bp) | 7 |
| PolyA sequence | 4553-4601 (49 bp) | 4567-4615 (49 bp) | 8 |
| Right AAV-ITR | 4624-4764 (141 bp) | 4638-4778 (141 bp) | 9 |

### Example 2. Unidirectional Promoter Tet-On Gene Expression Systems Have Tightly Controlled Expression and Reduced Basal Expression

Expression of SaCas9 was compared between the unidirectional promoter Tet-On expression systems (pCTX-277 and pCTX-279) described above in Example 1 and a conventional Tet-On system (pCTX-276) comprising a bidirectional promoter (Ftg83/TRE3GS) for simultaneous expression of Tet-On 3G transactivator protein and SaCas9 (see, construct design in FIG. 1). HEK293 cells (2×10⁶ cells) were transfected with 500 ng of one of the three plasmids and the cells were cultured for six days under standard condition (e.g., the culture medium was replaced every 24 hrs). Gene expression was induced by including 100 ng/mL of doxycycline (Dox) in the culture medium. SaCas9 protein expression was detected by Western blotting.

There was no detectable basal expression (i.e., leaky expression in the absence of Dox) of SaCas9 from pCTX-277 and pCTX-279, but there was detectable basal expression from pCTX-276, as shown in FIGS. 2A and 2B. Upon induction with Dox at day 3, the levels of SaCas9 expression from the expression cassette comprising the unidirectional promoter and the spacer (pCTX-279) was much higher than that from the other two expression systems. Upon removal of Dox, SaCas9 expression from the unidirectional promoter expression systems returned to basal levels within 1-2 days.

### Example 3. Increased Copy Number Increases Basal and Induced Expression

HEK293 cells (2×10⁶ cells) were transfected with 100 ng or 250 ng of each plasmid, and expression was monitored using an immunodetection system employing electrochemiluminescence (ECL) (e.g., a MSD platform). While Dox increased the level of SaCas9 expression in cells containing increased numbers of plasmids (FIGS. 3A and 3B), there was also increased basal expression from the unidirectional promoter systems in cells containing increased plasmid copy numbers (see FIG. 3B).

What is claimed is:

1. A nucleic acid comprising a unidirectional regulatable promoter operably linked to a transcription unit, the transcription unit encoding a protein of interest, a ribosome skip, and a transactivator protein.

2. The nucleic acid of claim 1, wherein the transcription unit comprises from 5′ to 3′ sequence encoding the protein of interest, sequence encoding the ribosome skip, and sequence encoding the transactivator protein.

3. The nucleic acid of claim 1 or 2, wherein the unidirectional regulatable promoter is a tetracycline-dependent promoter.

4. The nucleic acid of any one of claims 1 to 3, wherein the unidirectional regulatable promoter comprises a plurality of tetracycline operator (tetO) sequences located upstream of a minimal constitutive eukaryotic promoter.

5. The nucleic acid of claim 4, wherein the unidirectional regulatable promoter comprises from two to ten tetO sequences.

6. The nucleic acid of claim 4 or 5, wherein the minimal constitutive eukaryotic promoter is a minimal cytomegalovirus (CMV) promoter, a minimal elongation factor 1 (EF1) alpha promoter, or a minimal Simian virus 40 (SV40) promoter.

7. The nucleic acid of any one of claims 4 to 6, wherein the unidirectional regulatable promoter comprises seven tetO sequences located upstream of a minimal CMV promoter.

8. The nucleic acid of any one of claims 1 to 7, wherein the protein of interest is a recombinant protein or a therapeutic protein.

9. The nucleic acid of any one of claims 1 to 8, wherein the protein of interest encoded by the transcription unit is a CRISPR protein.

10. The nucleic acid of claim 9, wherein the CRISPR protein is a Cas9 protein, a Cpf1 protein, a Cas13 protein, a Cas14 protein, a CasX protein, or a CasY protein.

11. The nucleic acid of claim 9 or 10, wherein the CRISPR protein has less than about 1200 amino acids.

12. The nucleic acid of any one of claims 9 to 11, wherein the CRISPR protein is Staphylococcus aureus Cas9, Neisseria meningitidis Cas9, Campylobacter jejuni Cas9, or a variant having at least 90% sequence identity to said Cas9 protein.

13. The nucleic acid of any one of claims 9 to 12, wherein the CRISPR protein is a CRISPR nuclease, a CRISPR nickase, or a nuclease deficient CRISPR variant.

14. The nucleic acid of any one of claims 9 to 13, wherein sequence encoding the CRISPR protein is codon optimized for expression in a mammalian cell.

15. The nucleic acid of any one of claims 9 to 14, wherein the CRISPR protein is linked to at least one nuclear localization signal (NLS).

16. The nucleic acid of claim 15, wherein the at least one NLS is located at or within 50 amino acids of the amino terminus and/or at or within 50 amino acids of the carboxy terminus of the CRISPR protein.

17. The nucleic acid of any one of claims 1 to 16, wherein the ribosome skip encoded by the transcription unit is a 2A sequence family member.

18. The nucleic acid of any one of claims 1 to 17, wherein the transactivator protein encoded by the transcription unit is a variant of a reverse tetracycline transactivator (rtTA) protein that is linked to at least one activation domain.

19. The nucleic acid of claim 18, wherein sequence encoding the variant rtTA protein is codon optimized for expression in a mammalian cell.

20. The nucleic acid of claim 18 or 19, wherein the at least one activation domain is a VP16 activation domain or variant thereof.

21. The nucleic acid of claim 20, wherein the at least one activation domain comprises one or more repeats of a minimal VP16 activation domain.

22. The nucleic acid of any one of claims 18 to 21, wherein the transactivator protein comprises the variant rtTA protein linked to three repeats of a modified, minimal VP16 activation domain.

23. The nucleic acid of any one of claims 1 to 22, further comprising a polyadenylation signal sequence at its 3′ end.

24. The nucleic acid of any one of claims 1 to 23, further comprising an adeno-associated virus (AAV) inverted terminal repeat (ITR) at each end.

25. The nucleic acid of claim 24, further comprising a spacer between the AAV ITR at its 5′ end and the unidirectional regulatable promoter.

26. The nucleic acid of claim 25, wherein the spacer comprises from about 2 nucleotides or base pairs to about 30 nucleotides or base pairs.

27. The nucleic acid of any one of claims 1 to 26, wherein the transcription unit further encodes another ribosome skip and a fluorescent protein.

28. An expression cassette comprising the nucleic acid of any one of claims 1 to 27.

29. A vector comprising the expression cassette of claim 28.

30. A plasmid vector having a sequence as set forth in SEQ ID NO: 10.

31. A plasmid vector having a sequence as set forth in SEQ ID NO: 11.

32. An AAV particle comprising the nucleic acid of any one of claims 1 to 26 and at least one capsid protein.

33. A mammalian cell comprising the nucleic acid of any one of claims 1 to 27, the expression cassette of claim 28, the vector of any one of claims 29 to 31, or the AAV particle of claim 32.

34. A method for expressing a protein of interest in a cell, the method comprising
(a) introducing into the cell a regulatable expression system comprising a unidirectional regulatable promoter, wherein the regulatable expression system is provided by the nucleic acid of any one of claims 1 to 27, the expression cassette of claim 28, the vector of any one of claims 29 to 31, or the AAV particle of claim 32, and
(b) exposing the cell to a promoter regulating agent.

35. The method of claim 34, wherein the promoter regulating agent is doxycycline.

36. The method of any one of claim 34 or 35, wherein basal expression of the protein of interest from the regulatable expression system comprising a unidirectional regulatable promoter is less than that from a regulatable expression system comprising a bidirectional regulatable promoter.

37. The method of any one of claims 34 to 36, wherein, upon exposure to the promoter regulating agent, expression of the protein of interest from the regulatable expression system comprising a unidirectional regulatable promoter is increased as compared to that from a regulatable expression system comprising a bidirectional regulatable promoter.

38. The method of any one of claims 34 to 37, wherein, upon exposure to the promoter regulating agent, expression of the protein of interest from the regulatable expression system comprising a unidirectional regulatable promoter is increased by at least 10-fold over basal expression.

39. The method of any one of claims 34 to 38, wherein the protein of interest is a CRISPR protein.

40. The method of claim 39, wherein the CRISPR protein is a Cas9 protein, a Cpf1 protein, a Cas13 protein, a Cas14 protein, a CasX protein, or a CasY protein.

41. The method of claim 39 or 40, wherein the CRISPR protein has less than about 1200 amino acids.

42. The method of any one of claims 39 to 41, wherein the CRISPR protein is Staphylococcus aureus Cas9, Neisseria meningitidis Cas9, Campylobacter jejuni Cas9, or a variant having at least 90% sequence identity to said Cas9 protein.
